(19) United States
(12) Patent Application Publication
(10) Pub. No.: US 2008/0008704 A1
(43) Pub. Date: Jan. 10, 2008

(54) Methods of treating colorectal cancer with anti-epidermal growth factor antibodies

(76) Inventor: Mark Rubin, Bonita Springs, FL (US)

Correspondence Address: KENYON & KENYON LLP, ONE BROADWAY, NEW YORK, NY, 10004, US

(21) Appl. No.: 11/732,204

(22) Filed: Apr. 2, 2007

Related U.S. Application Data
(63) Continuation of application No. 09/809,924, filed on Mar. 16, 2001, now abandoned.

(51) Int. Cl. A61K 39/395; A61P 35/00

(52) U.S. Cl. 424/133.1; 424/142.1; 424/143.1

(57) ABSTRACT

A method for treating colorectal cancer is disclosed and consists of administering to a patient in need of such treatment a pharmaceutically effective dose of an agent capable of blocking the binding of EGF-receptor to its natural ligand(s) and/or inhibiting EGF-receptor-mediated signaling. Also disclosed is a method of increasing a colorectal cancer patient response to anti-cancer therapy which consists of administering to said patient a pharmaceutically effective dose of an agent capable of blocking the binding of EGF-receptor to its natural ligand(s) and/or inhibiting EGF-receptor-mediated signaling.

Throughout this application, various references are referred to. Disclosures of these publications in their entireties are hereby incorporated by reference into this application to more fully describe the state of the art to which this invention pertains.

### FIELD OF THE INVENTION

The present invention relates to the treatment of colorectal cancer by inhibiting epidermal growth factor (EGF) receptor-mediated signaling.

### DESCRIPTION OF THE RELATED ART

Growth factor receptor tyrosine kinases play an important role in the etiology and progression of human malignancies. These biological receptors are anchored by means of a transmembrane domain in the membranes of cells that express them. An extracellular domain binds to a growth factor. The binding of the growth factor to the extracellular domain results in a signal being transmitted to the intracellular kinase domain. The transduction of this signal contributes to the events that are responsible for the proliferation and differentiation of the cells.

Members of the epidermal growth factor (EGF) receptor family are important growth factor receptor tyrosine kinases. The first member of the EGF receptor family to be discovered was named the EGF receptor which is a glycoprotein having an apparent molecular weight of approximately 165 kD (U.S. Pat. No. 4,943,533).

The binding of an EGF receptor ligand to the EGF receptor leads to cell growth. EGF and transforming growth factor alpha (TGF-alpha) are two known ligands of EGF receptor.

Many receptor tyrosine kinases are found in unusually high numbers on human tumors. For example, many tumors of epithelial origin express increased levels of EGF receptor on their cell membranes. Examples of tumors that express EGF receptors include glioblastomas, as well as cancers of the lung, breast, head and neck, and bladder. The amplification and/or overexpression of the EGF receptors on the membranes of tumor cells is associated with a poor prognosis.

Antibodies, especially monoclonal antibodies, raised against tumor antigens have been investigated as potential anti-tumor agents. Such antibodies may inhibit the growth of tumors through a number of mechanisms. For example, antibodies may inhibit the growth of tumors immunologically through antibody-dependent cellular cytotoxicity (ADCC) or complement-dependent cytotoxicity (CDC).

Alternatively, antibodies may compete with growth factors in binding to their receptor. Such competition inhibits the growth of tumors that express the receptor.

In another approach, toxins are conjugated to antibodies raised against tumor antigens. The antibody portion directs the conjugate to the tumor, which is killed by the toxin portion.

For example, U.S. Pat. No. 4,943,533 describes a murine monoclonal antibody called M225 (a murine antibody) that binds to the EGF receptor. The M225 antibody is able to inhibit the growth of cultured EGF receptor-expressing tumor lines as well as the growth of thee tumors in vivo when grown as xenografts in nude mice. In mouse xenograft models, in vivo experiments of twice weekly intra-peritoneal injection were investigated based on a measured monoclonal antibody half-life in serum of three days (Masui et al., Cancer Res. 44:1002-1007, 1984). Administration of M225 monoclonal antibody beginning concurrent with human subcutaneous tumor cell implantation, caused a dose dependent suppression of A431 squamous cells (Masui et al., Cancer Res. 44:1002-1007, 1984), MDA-468 breast carcinoma (Mendelsohn, Cancer Cells, 359:362, 1989), and DiFi colon carcinomas (Masui et al., Proc. Amer. Assoc. Cancer Res., 32:394, 1991). For most well established tumors (10 to 20 days), treatment with anti-EGFr monoclonal antibody produced varying degrees of cytostasis, without eliminating the tumors (Baselga et al., J. Natl. Cancer Inst. 85:1327-1333, 1993); Fan et al., Cancer Res. 53:4637-4642, 1993; Masui et al., Cancer Res. 44:1002-1007, 1984).

In a phase I clinical trial, however, no clinical response was observed when up to 300 mg of M225 antibodies were administered to humans (Divgi et al., J. Natl. Cancer Inst. 83:97-104, 1991; Masui et al., Cancer Res. 44:5592-5598, 1986).

A disadvantage of using murine monoclonal antibodies in human therapy is the possibility of a human anti-mouse antibody (HAMA) response due to the presence of mouse Ig sequences. This disadvantage can be minimized by replacing the entire constant region of a murine (or other non-human mammalian) antibody with that of a human constant region. Replacement of the constant regions of a murine antibody with human sequences is usually referred to as chimerization (human:murine chimeric version). The chimeric equivalent of M225 is C225 (also named CETUXIMAB® by Imclone Systems Incorporated) anti-EGF receptor antibody.

A spectrum of human squamous cell carcinoma cell lines have now been studied which confirm the capacity of C225 to modulate tumor cell proliferation, cell cycle phase distribution, apoptosis and radiosensitivity (Huang et al., Cancer Res., 59:1935-1940, 1999; Huang et al., American Association for Cancer Research, 89^{th} Annual Meeting. New Orleans, La., 1998; Ciardiello et al., Clinical Cancer research, 4:909-916, 1999). However, clinical trials with C225 have given mixed results. For instance, while a percentage of patients suffering from head and neck tumors at least partially responded to treatment in particular when combined with radiation or chemotherapy, other patients such as breast cancer patients showed no response even when the breast cancer cells showed strong expression of EGF receptors. Based on those results, it is not possible to predict an in vivo response to various kinds of cancers including colon cancer where a need for a new form of successful therapy has existed for a long time.

In 1998, an estimated 131,600 people in the United States were diagnosed with colon cancer. Sixty percent of these patients presented with Stage II or III diseases. Of that group, approximately 35% to 40% of them will experience recurrence of metastatic or locally invasive disease. The majority of these recurrences in patients who have undergone a complete resection of a colorectal cancer will occur within FIVE years, and usually within three years of surgery (Desch et al., J. Clin. Onc., 17(4):1312-1321, 1999).

Inoperable metastatic colon cancer is incurable. There is no standard chemotherapy for patients with widespread metastatic disease, but trials with 5-fluorouracil (5-FU) and LEUCOVORIN® have demonstrated increased numbers of partial responses and prolongation of the time to progression of disease (Petrelli et al., J. Clin. Onc., 5(10):1559-1565, 1987), as well as improved survival and quality of life for patients receiving chemotherapy compared to best supportive care (Scheithauer et al., Brit. Med. J., 306(6680):752-755, 1993). Patients should be considered candidates for clinical trials evaluating new approaches to treatment (Moertel, New Eng. J. Med., 330(16):1136-1142, 1994; Poon et al., J. Clin. Onc., 9(11):1967-1976, 1991). IRINOTECAN® (CPT-11) is a topoisomerase-I inhibitor with a 10% to 20% partial response rate in patients with metastatic colon cancer, in patients who have received no prior chemotherapy, and in patients progressing on 5-FU® therapy (Conti et al., J. Clin. Onc., 14(3):709-715, 1996; Rothenberg et al., J. Clin. Onc., 14(4):1128-1135, 1996). It has been approved by the FDA for the treatment of patients with metastatic disease that is refractory to 5-FU. However, despite these new approaches metastatic colon cancer remains refractory to most forms of chemotherapy.

### SUMMARY OF THE INVENTION

The present invention provides a method for treating colorectal cancer comprising administering to a patient in need of such treatment a pharmaceutically effective dose of an agent capable of blocking the binding of EGF receptor to its natural ligand(s) and/or inhibiting EGF receptor-mediated signaling.

The present invention also provides a method of increasing a colorectal cancer patient response to anti-cancer therapy comprising administering to said patient a pharmaceutically effective dose of an agent capable of blocking the binding of EGF receptor to its natural ligand(s) and/or inhibiting EGF receptor-mediated signaling.

### DETAILED DESCRIPTION OF THE INVENTION

Surprisingly, It has now been found that colorectal cancer can be treated by the administration of a pharmaceutically effective dose of an agent capable of blocking the binding of EGF receptor to its natural ligand(s) and/or inhibiting EGF receptor-mediated signaling. It has also been surprisingly found that a colorectal cancer patient response to anti-cancer therapy is increased by administering to said patient a pharmaceutically effective dose of an agent capable of blocking the binding of EGF receptor to its natural ligand(s) and/or inhibiting EGF receptor-mediated signaling.

Preferably, the colorectal cancer is colon cancer. Also, preferably, the colorectal cancer expresses cell-surface EGF receptors.

The agent used in the treatment is preferably is an anti EGF-receptor antibody, a fragment thereof, a single chain antibody, more preferably a monoclonal antibody and most preferably a humanized (human:murine chimeric antibody or humanized antibody) comprising a non-human variable and/or hypervariable region and a human constant region. The antibody may also be a full human antibody. The production of single chain, humanized and chimeric anti-EGF receptor antibodies is well known in the art (U.S. Pat. Nos. 5,558,864 and 5,844,093).

Alternatively, the agent used in the treatment can be any inhibitor of tyrosine kinase activity mediated by EGF receptors such as IRESSA® or any agent or compound that interferes with the binding of the EGF receptor to its natural ligands (i.e., EGF and TGF-alpha). For example, soluble forms of EGF receptors having the extracellular domain of EGF receptors could compete with EGF receptors for binding with EGF and TGF-alpha thus acting as inhibitors for the activation of EGF receptors.

Irradiation therapy can be conducted and/or a pharmaceutically effective dose of at least one chemotherapy agent can be administered before, during and/or after the administration of the agent capable of blocking the binding of EGF receptor to its natural ligand(s) and/or inhibiting EGF receptor-mediated signaling. Also preferably, the chemotherapy agent is selected from the group consisting of IRITONECAN® (CPT-11), 5-florouracil (5-FU), CISPLATIN® (CDDP), OXALOPLATIN®, LEUCOVORIN® and BRYOSTATIN®, most preferably IRITONECAN®.

As indicated above, the use of the chimeric monoclonal anti-EGF receptor antibody C225 showed some success in inhibiting cancer growth in vitro and in human xenograft models in mice. However, the same degree of success was not translated in human clinical trials even on cancer cells that strongly express the cell surface EGF receptor and even when concurrent chemotherapy was used. For instance, compassionate clinical trials using C225 on patients suffering from head and neck squamous carcinomas resulted in at least a partial response in some patients. However, the treatment with C225 of several patients suffering from breast, renal, tongue, esophagus, nasopharyngeal, pancreatic, prostate, cervical and larynx cancer resulted in no positive response (a stable disease is not considered here as a positive response). This made it impossible to predict human clinical success even when positive in vitro or xenograft models data were available. Thus, the present finding that C225 antibodies were effective in the treatment of colon cancer is indeed surprising and not predictable. These results outlined below clearly indicate that blocking the binding of EGF receptor to its natural ligand(s) and/or inhibiting EGF receptor-mediated signaling can be successfully used in the treatment of colon cancer.

It is noteworthy that a person skilled in the art would understand that dosages and frequency of treatment vary depending on the tolerance of the individual patient and on the pharmacological and pharmacokinetic properties of each blocking or inhibitory agent used. Ideally, one wishes to achieve saturable pharmacokinetics for the agent used. When antibodies are used as blocking agents or inhibitors of the present invention, they can be administered to patients in any one of conventional ways well known to people skilled in the art. For instance, antibodies such as C225 in a pharmaceutical composition can be administered intravenously. A loading dose (i.e., initial dose) can range for example, from about 10 to 1000 mg/m², preferably about 200 to 400 mg/m². This is followed by several additional daily or weekly dosages raging for example, from about 200 to 400 mg/m². The patient is closely monitored for side effects such as skin toxicity and the treatment is stopped when such side effects are severe.

As indicated above, radiation or chemotherapy treatment may be employed before, during or after the use of the blocking agents or inhibitors of the present invention. Protocols using numerous irradiation treatments and/or chemotherapy agents are well known in the art (Rothenberg et al., J Clin. Onc., 14(4):1128-1135, 1996). Preferably, the chemotherapy agent(s) used is the one that normally is the most effective for the particular case. For example, IRITONECAN® is a preferred agent since it was found to have significant single-agent activity against colorectal cancer (Rothenberg et al., J Clin. Onc., 14(4):1128-1135, 1996).

### EXAMPLE 1

A female patient presented with abdominal pain and constipation. Colonoscopy showed a bulky ulcerated colonic neoplasm. A right hemicolectomy was undertaken and the patient was found to have a mass in distal right colon as well as omental metastasis and liver metastasis. Pathology confirmed metastatic colon cancer which is incurable. She was treated on a clinical trial with oral 5-FU but progressed with increasing liver metastasis (2 lesions). Subsequently, she was treated with CPT-11 but continued to progress in the liver and was enrolled on a second clinical trial with OXALOPLATIN® which resulted in further disease progression in the liver. Since there were no other standard treatment options her tumor was assayed for EGF receptor which was positive. Therefore, she was offered treatment with C225 and CPT-11. Just prior to treatment with C225 and CPT-11, lesions in the liver measured 65 cm² and 45 cm² and CEA levels were at 1,231 ng/ml.

The patient was treated with C-225, loading dose of 400 mg/m². A course of therapy was defined as four infusions of CETUXIMAB® (C225).

After receiving one course of four weekly consecutive doses of C225 and three doses of IRINOTECAN®, the patient showed an average of 57.5% reduction in tumor size. For the first two weeks, IRINOTECAN® was administered at a dose of 125 mg/m². The Week 3 dose was held because of diarrhea and the Week 4 dose was reduced to 94 mg/m². The Week 4 dose was further reduced to 69 mg/m² because of neutropenia. There were no dose reductions of C225. In addition, after 4 doses of C225, the patient's baseline CEA was dramatically reduced to 332 ng/ml.

Maximal response was achieved at 4 months after initial treatment with lesions measuring 12 cm² and 10 cm². Performance status was maintained at 90% (she worked full time) and her QOL was excellent, continuing normal activities. Response was durable for about 7 to 9 months which allowed for surgical resection of residual liver disease. It is important to note that prior to treatment the patient had unresectable liver disease. C225 was discontinued prior to surgery. Following resection she had no evidence of disease.

This result clearly indicates that C225 can be used as an effective treatment at least in some patients against colorectal cancer.

### EXAMPLE 2

The subsequent history of the patient described in Example 1 is as follows. The patient remained off of C225 and chemotherapy and 4 weeks after surgery, she relapsed with an ovary metastasis. This was resected and subsequently she received both intrahepatic and systemic chemotherapy (FUDR and CPT-11) for 6 months. During that time, she did not receive C225 therapy. At the completion of treatment, she relapsed with a rising CEA. An abdominal exploration was undertaken and she was found to have unresectable peritoneal disease. In addition, she had multiple pulmonary metastasis. There were no further treatment options available and therefore, she was again offered C225. She was treated with a combination of C225 and dose reduced CPT-11 with the CEA decreasing to normal (CEA=4) from pretreatment level of 43. Abdominal CAT SCAN (CT) showed no signs of progression following treatment and the quality of life improved. Subsequently, chemotherapy was stopped with C225 being administered alone. CPT-11 was discontinued secondary to toxicity but response has been maintained on C225 alone for at least up to 2 months.

The invention has been described in terms of preferred embodiments and examples, but is not limited thereby. Those of skill in the art will readily recognize the broader applicability and scope of the invention which is limited only by the patent claims herein.

What is claimed is:

1. A method for treating colorectal cancer comprising administering to a patient in need of such treatment a pharmaceutically effective dose of an agent capable of blocking the binding of EGF receptor to its natural ligand(s) and/or inhibiting EGF receptor-mediated signaling.

2. The method of claim 1, wherein the cancer is colon cancer.

3. The method of claim 1, wherein the colorectal cancer expresses cell-surface EGF receptors.

4. The method of claim 1, wherein said agent is an anti EGF-receptor antibody or a fragment thereof.

5. The method of claim 4, wherein the antibody is a monoclonal antibody or a fragment thereof.

6. The method of claim 4, wherein the antibody is a chimeric antibody.

7. The method of claim 6, wherein the antibody is C225.

8. The method of claim 6, wherein the chimeric antibody comprises a non-human variable and/or hypervariable region and a human constant region.

9. The method of claim 4, wherein the antibody is a human antibody.

10. The method of claim 1 further comprising administering to said patient a pharmaceutically effective dose of at least one chemotherapy agent before, during and/or after the administration of the agent capable of blocking the binding of EGF receptor to its natural ligand(s) and/or inhibiting EGF receptor-mediated signaling.

11. The method of claim 10, wherein the chemotherapy agent is selected from the group consisting of IRITONECAN® (CPT-11), 5-florouracil (5-FU), CISPLATIN® (CDDP), OXALOPLATIN®, LEUCOVORIN® and BRYOSTATIN®.

12. The method of claim 11, wherein the chemotherapy agent is IRITONECAN®.

13. The method of claim 1 further comprising having said patient undergo irradiation therapy before, during and/or after the administration of the agent capable of blocking the binding of EGF receptor to its natural ligand(s) and/or inhibiting EGF receptor-mediated signaling.

14. A method of increasing a colorectal cancer patient response to anti-cancer therapy comprising administering to said patient a pharmaceutically effective dose of an agent capable of blocking the binding of EGF receptor to its natural ligand(s) and/or inhibiting EGF receptor-mediated signaling.

15. The method of claim 14, wherein the cancer is colon cancer.

16. The method of claim 14, wherein the colorectal cancer expresses cell-surface EGF receptors.

17. The method of claim 14, wherein said agent is an anti EGF-receptor antibody or a fragment thereof.

18. The method of claim 17, wherein the antibody is a monoclonal antibody or a fragment thereof.

19. The method of claim 17, wherein the antibody is a chimeric antibody.

20. The method of claim 19, wherein the antibody is C225.

21. The method of claim 19, wherein the chimeric antibody comprises a non-human variable and/or hypervariable region and a human constant region.

22. The method of claim 17, wherein the antibody is a human antibody.

23. The method of claim 14, wherein the anti-cancer therapy is chemotherapy.

24. The method of claim 14 further comprising administering to said patient a pharmaceutically effective dose of at least one chemotherapy agent before, during and/or after the administration of the agent capable of blocking the binding of EGF receptor to its natural ligand(s) and/or inhibiting EGF receptor-mediated signaling.

25. The method of claim 23, wherein the chemotherapy agent is selected from the group consisting of IRITONECAN® (CPT-11), 5-florouracil (5-FU), CISPLATIN® (CDDP), OXALOPLATIN®, LEUCOVORIN® and BRYOSTATIN®.

26. The method of claim 25, wherein the chemotherapy agent is IRITONECAN® (CPT-11).

27. The method of claim 14, wherein the anti-cancer therapy is irradiation therapy.
